(19) United States
(12) Patent Application Publication
(10) Pub. No.: US 2006/0058610 A1
(43) Pub. Date: Mar. 16, 2006

(54) Increasing the efficiency of quantitation in stress echo

(75) Inventor: Bjorn Olstad, Stathelle (NO)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/087,277

(22) Filed: Mar. 22, 2005

Related U.S. Application Data
(60) Provisional application No. 60/605,953, filed on Aug. 31, 2004.

(51) Int. Cl. A61B 5/05

(52) U.S. Cl. 600/407

(57) ABSTRACT

The present invention relates to a method and apparatus for extracting ultrasound summary information useful for increasing efficiency of quantitation of a stress echo examination performed using an ultrasound machine. One embodiment of the present invention comprises a front-end arranged to transmit ultrasound waves into moving cardiac structure and blood and generate received signals in response to the ultrasound waves backscattered from moving cardiac structure and blood. At least one processor responsive to the received signals identifies at least one anatomical landmark within the heart, generates a report based at least in part on one key parameter extracted from the anatomical landmark, and scores the at least one extracted parameter. The anatomical landmarks, reports, and scores may be displayed to a user.

### RELATED APPLICATIONS/INCORPORATION BY REFERENCE

This application is related to, and claims benefit of and priority from, Provisional Application No. 60/605,953, filed Aug. 31, 2004, titled “INCREASING THE EFFICENCY OF QUANTITATION IN STRESS ECHO”, the complete subject matter of which is incorporated herein by reference in its entirety.

The complete subject matter of each of the following U.S. patent applications is incorporated by reference herein in their entirety:
U.S. patent application Ser. No. 10/248,090 filed on Dec. 17, 2002.
U.S. patent application Ser. No. 10/064,032 filed on Jun. 4, 2002.
U.S. patent application Ser. No. 10/064,083 filed on Jun. 10, 2002.
U.S. patent application Ser. No. 10/064,033 filed on Jun. 4, 2002.
U.S. patent application Ser. No. 10/064,084 filed on Jun. 10, 2002.
U.S. patent application Ser. No. 10/064,085 filed on Jun. 10, 2002.
U.S. patent application Ser. No. 60/605,939, filed on Aug. 31, 2004.

### FEDERALLY SPONSORED RESEARCH OR DEVELOPMENT

[Not Applicable]

### BACKGROUND OF THE INVENTION

Embodiments of the present invention relate to an ultrasound system. More specifically, embodiments of the present invention relate to extracting ultrasound summary information useful for increasing efficiency of quantitation of a stress echo examination performed using an ultrasound machine.

Echocardiography is a branch of the ultrasound field that is currently a mixture of subjective image assessment and extraction of key quantitative parameters. Evaluation of cardiac function has been hampered due to a lack of well-established parameters that may be used to increase the accuracy and objectivity in the assessment of diseases (coronary artery diseases for example). It has been shown that inter-observer variability between echo-centers is unacceptably high due to the subjective nature of the cardiac motion assessment.

Technical and clinical research has focused on this problem, aimed at defining and validating quantitative parameters. Encouraging clinical validation studies indicate a set of new potential quantative parameters that may be used to increase objectivity and accuracy in the diagnosis of coronary artery diseases for example. It has been found that many of the new parameters have been difficult or impossible to assess by direct visual inspection of the ultrasound images generated in real-time. The quantification of these parameters has typically required a post-processing step using tedious, manual analysis to extract the necessary parameters. Such analyses usually requires manually localizing anatomical landmarks and extracting parameters (such as velocity or strain-rate profiles for example) at these locations. Time intensive post-processing techniques or complex, computation-intensive real-time techniques are undesirable. It is contemplated that improving the efficiency of the quantative process would be greatly desired.

One method disclosed in U.S. Pat. No. 5,601,084 to Sheehan et al. describes imaging and three-dimensionally modeling portions of the heart using imaging data. Another method disclosed in U.S. Pat. No. 6,099,471 to Torp et al. describes calculating and displaying strain velocity in real time. Still another method disclosed in U.S. Pat. No. 5,515,856 to Olstad et al. describes generating anatomical M-mode displays for investigations of living biological structures, such as heart function, during movement of the structure. Yet another method disclosed in U.S. Pat. No. 6,019,724 to Gronningsaeter et al. describes generating quasi-real-time feedback for the purpose of guiding procedures by means of ultrasound imaging.

### BRIEF SUMMARY OF THE INVENTION

One embodiment of the present invention relates to increasing the efficiency of quantitation in stress echo. One embodiment of the present invention relates to extracting ultrasound summary information (using an ultrasound machine for example) useful for increasing the efficiency of quantitation in stress echo examination.

One embodiment of the present invention relates to an ultrasound system for imaging a heart and extracting ultrasound summary information useful for increasing the efficiency of quantitation in stress echo. This embodiment comprises a front-end arranged to transmit ultrasound waves into moving cardiac structure and blood and generate received signals in response to the ultrasound waves backscattered from moving cardiac structure and blood. At least one processor responsive to the received signals identifies at least one anatomical landmark within the heart, generates a report based at least in part on one key parameter extracted from the anatomical landmark, and scores the at least one extracted parameter. The anatomical landmarks, reports, and scores may be displayed to a user.

One embodiment relates to a method for assessing an image responsive to moving cardiac structure and blood within a heart of a subject. This embodiment comprise identifying at least one anatomical landmark within the heart. A report is generated based at least in part on one key parameter extracted from the anatomical landmark and at least the one extracted parameter is scored.

One embodiment of the present invention relates to an ultrasound machine for generating an image responsive to moving cardiac structure and blood within a heart of a subject. This method comprises acquiring at least one apical view of the heart using the ultrasound machine, and generating and displaying at least one image of the apical view. This method further comprises automatically identifying at least one anatomical landmark from at least one of the views using the ultrasound machine, generating a report based at least in part on the at least one key parameter and scoring the at least one extracted parameter.

Certain embodiments of the present invention afford an approach to extract certain clinically relevant information from a heart after automatically locating key anatomical landmarks of the heart, such as the apex and the AV-plane.

### BRIEF DESCRIPTION OF THE DRAWINGS

The foregoing summary, as well as the following detailed description of certain embodiments of the present invention, will be better understood when read in conjunction with the appended drawings. It should be understood, however, that the present invention is not limited to the arrangements and instrumentality shown in the attached drawings.

FIG. 1 depicts a block diagram of an embodiment of an ultrasound machine made in accordance with various embodiments of the present invention.

FIGS. 2A and 2B depict flowcharts illustrating an embodiment of a method of performing efficient quantitative analysis of stress echoes performed using embodiments of the machine illustrated in FIG. 1, in accordance with various embodiments of the present invention.

FIG. 3 depicts using the method of FIGS. 2A and 2B to identify the lower parts of basal segments and mid segments within a heart in accordance with embodiments of the present invention.

FIG. 4 depicts an apical 4-chamber view of a heart illustrating normal peak values at peak exercise in dobutamine stress in accordance with embodiments of the present invention.

FIG. 5 depicts an apical 2-chamber view of the heart (similar to the diagram of FIG. 4) illustrating normal peak values at peak exercise in dobutamine stress in accordance with embodiments of the present invention.

FIG. 6 depicts response curves for peak systolic velocities as a function of dobutamine level in a normal population in accordance with embodiments of the present invention.

FIG. 7 depicts one known method used to acquire and display a stress echo using an ultrasound machine in accordance with the present invention.

FIG. 8 depicts one method for detecting the AV-plane using the method of FIGS. 2A and 2B to extract at least one key parameter and generate a basis for reporting using an ultrasound device in accordance with embodiments of the present invention.

FIG. 9 depicts one method for detecting the AV-plane using the method of FIGS. 2A and 2B to extract at least one key parameter and generate a basis for reporting (similar to that depicted in FIG. 8) in accordance with embodiments of the present invention.

FIG. 10 depicts one method for detecting the AV-plane using the method of FIGS. 2A and 2B to extract at least one key parameter and generate a basis for reporting in accordance with embodiments of the present invention.

FIG. 11 depicts one method for scoring subjective wall motion.

FIG. 12 depicts a method for augmenting subjective wall motion assessment using quantitative scoring in accordance with embodiments of the present invention.

FIG. 13 depicts one method for displaying information about normal ranges of one or more key parameters in accordance with embodiments of the invention.

FIG. 14 depicts one method for performing quantitative wall motion scoring in accordance with one or more embodiments of the present invention.

FIG. 15 depicts one method for using a scoring screen to review quantitative scoring in accordance with one or more embodiments of the present invention.

FIG. 16 depicts one method for automatically performing wall motion scoring in accordance with one or more embodiments of the present invention.

### DETAILED DESCRIPTION OF THE INVENTION

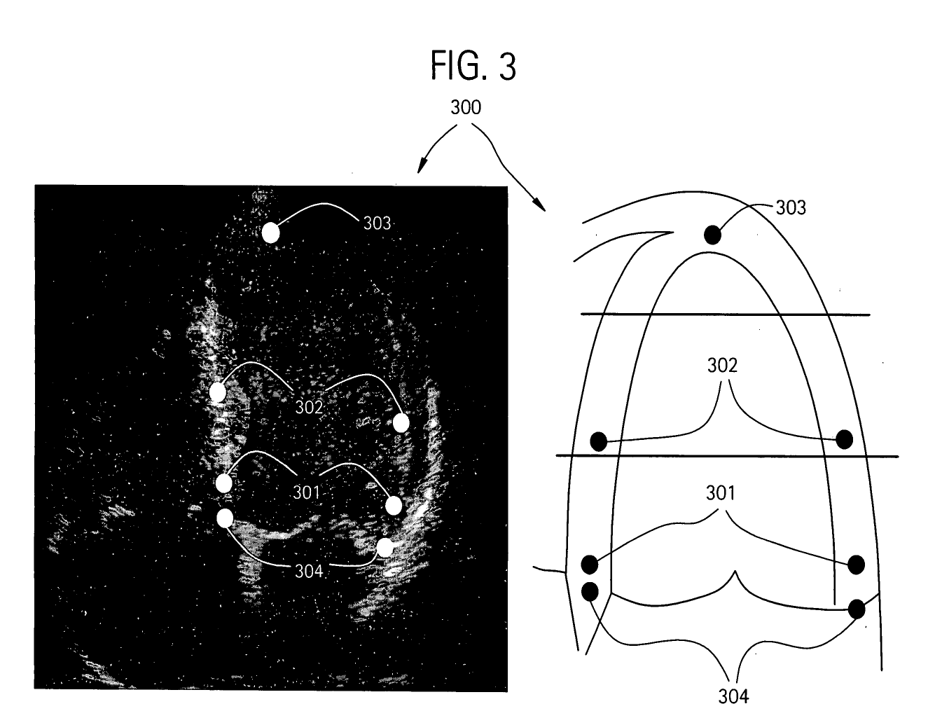

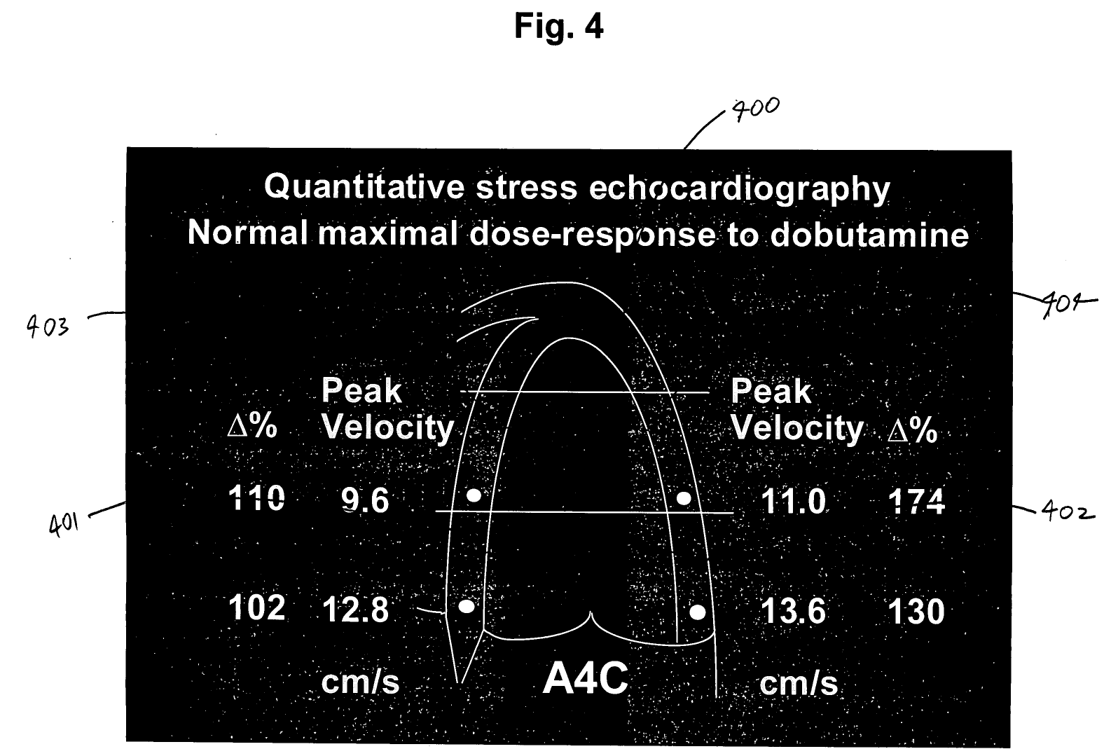

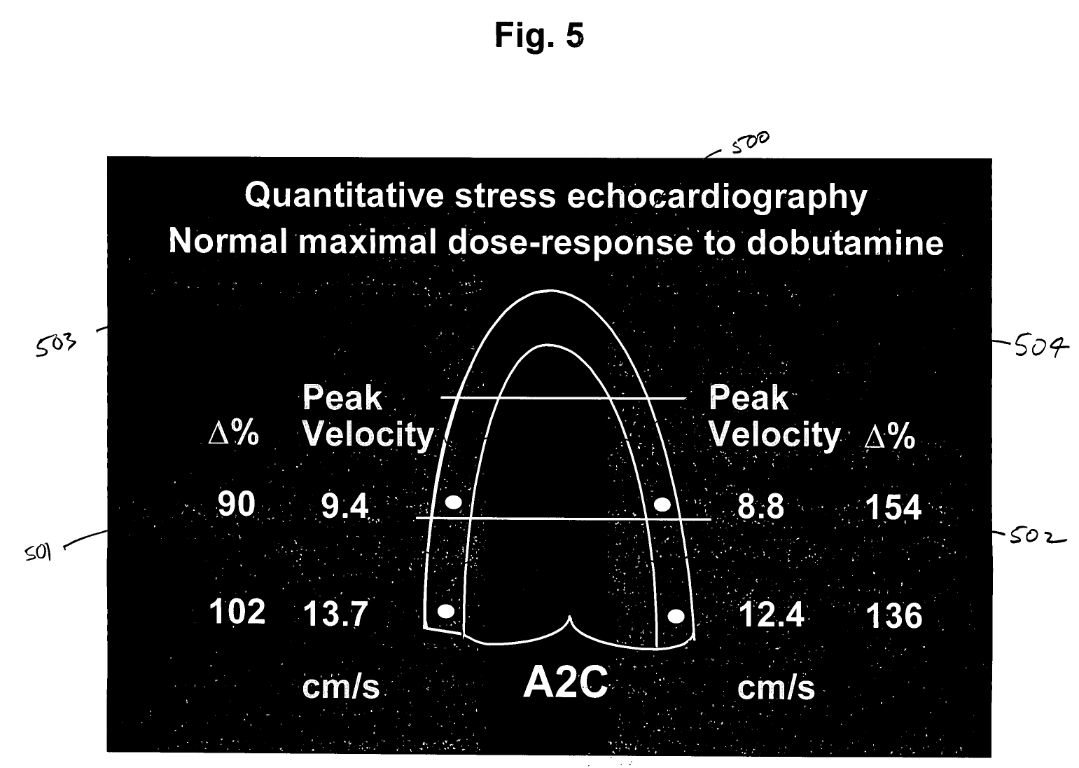

An embodiment of the present invention enables efficiently extraction of quantative information from a stress echo examination. Another embodiment of the present invention enables efficient extraction of quantative information from within a heart after locating and tracking certain anatomical landmarks of the heart using an ultrasound machine or device for example. Moving cardiac structure is monitored to accomplish this function. As used herein, the term structure comprises non-liquid and non-gas matter, such as cardiac tissue for example. An embodiment of the present invention provides improved, real-time visualization and quantative assessment of certain clinically relevant parameters of the heart. The moving structure is characterized by a set of analytic parameter values corresponding to anatomical points within a myocardial segment of the heart. The set of analytic parameter values may comprise, for example, tissue velocity values, time-integrated tissue velocity values, B-mode tissue intensity values, tissue strain rate values, blood flow values, tissue movement, strain rate, aggregated strain (including relative measurements between two points or across a region and mitral valve inferred values. Throughout the application and the figures, at least one embodiment extracts one or more parameters from one or more anatomical landmarks. It should be understood that, in at least one embodiment, such extraction process is understood to include a measurement of parameters across a localized region of interest responsive to the location of the anatomical landmark. Examples of such measurements include, but are not limited to: point or regional measurements at the anatomical landmark; point or regional measurements at locations computed relative to the anatomical landmark as illustrated in FIGS. 3, 4 and 5, where measurements in the lower basal segments are performed based on the AV-plane landmarks; point or regional measurements sampled relative to multiple anatomical landmarks as illustrated in FIGS. 3, 4 and 5, where measurements in the lower mid segment are performed based on the location of the AV-plane and apex landmarks; point or regional measurements sampled relative to one or multiple landmarks combined with geometrical shapes as illustrated in FIGS. 4 and 5, where the AV-plane and apex landmarks combined with the geometrical shape of the endocardial border is used to determine a multitude of sampling regions along the cardiac wall.

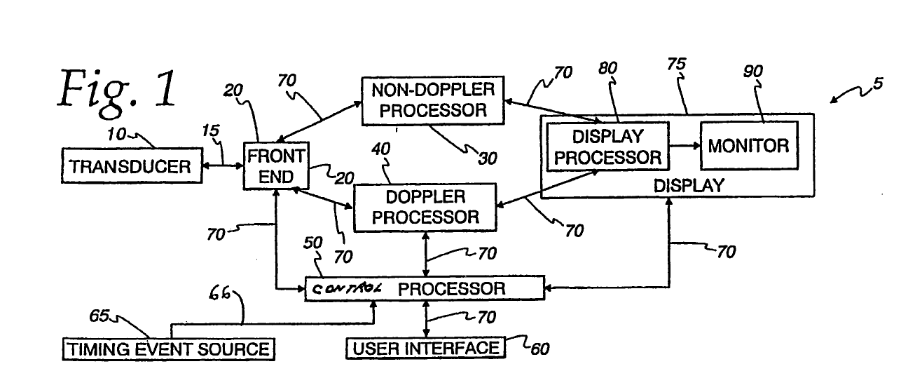

FIG. 1 illustrates an embodiment of an ultrasound machine, generally designated 5, in accordance with embodiments of the present invention. A transducer 10 transmits ultrasound waves into a subject by converting electrical analog signals to ultrasonic energy and receives the ultrasound waves backscattered from the subject by converting ultrasonic energy to analog electrical signals. A front-end 20, that in one embodiment comprises a receiver, transmitter, and beamformer, may be used to create the necessary transmitted waveforms, beam patterns, receiver filtering techniques, and demodulation schemes that are used for the various imaging modes. Front-end 20 performs such functions, converting digital data to analog data and vice versa. Front-end 20 interfaces to transducer 10 using analog interface 15 and interfaces to a non-Doppler processor 30, a Doppler processor 40 and a control processor 50 over a bus 70 (digital bus for example). Bus 70 may comprise several digital sub-buses, each sub-bus having its own unique configuration and providing digital data interfaces to various parts of the ultrasound machine 5.

Non-Doppler processor 30 is, in one embodiment, adapted to provide amplitude detection functions and data compression functions used for imaging modes such as B-mode, M-mode, and harmonic imaging. Doppler processor 40, in one embodiment provides clutter filtering functions and movement parameter estimation functions used for imaging modes such as tissue velocity imaging (TVI), strain rate imaging (SRI), and color M-mode. In one embodiment, the two processors, 30 and 40, accept digital signal data from the front-end 20, process the digital signal data into estimated parameter values, and pass the estimated parameter values to processor 50 and a display 75 over digital bus 70. The estimated parameter values may be created using the-received signals in frequency bands centered at the fundamental, harmonics, or sub-harmonics of the transmitted signals in a manner known to those skilled in the art.

Display 75 is adapted, in one embodiment, to provide scan-conversion functions, color mapping functions, and tissue/flow arbitration functions, performed by a display processor 80 which accepts digital parameter values from processors 30, 40, and 50, processes, maps, and formats the digital data for display, converts the digital display data to analog display signals, and communicate the analog display signals to a monitor 90. Monitor 90 accepts the analog display signals from display processor 80 and displays the resultant image.

A user interface 60 enables user commands to be input by the operator to the ultrasound machine 5 through control processor 50. User interface 60 may comprise a keyboard, mouse, switches, knobs, buttons, track balls, foot pedals, voice control and on-screen menus, among other devices.

A timing event source 65 generates a cardiac timing event signal 66 that represents the cardiac waveform of the subject. The timing event signal 66 is input to ultrasound machine 5 through control processor 50.

In one embodiment, control processor 50 comprises the central processor of the ultrasound machine 5, interfacing to various other parts of the ultrasound machine 5 through digital bus 70. Control processor 50 executes the various data algorithms and functions for the various imaging and diagnostic modes. Digital data and commands may be communicated between control processor 50 and other various parts of the ultrasound machine 5. As an alternative, the functions performed by control processor 50 may be performed by multiple processors, or may be integrated into processors 30, 40, or 80, or any combination thereof. As a further alternative, the functions of processors 30, 40, 50, and 80 may be integrated into a single PC backend.

Once certain anatomical landmarks of the heart are identified, (e.g., the AV-planes and apex as described in U.S. patent application Ser. No. 10/248,090 filed on Dec. 17, 2002) certain relevant summary information (key parameters for example) may be extracted and displayed to a user of the ultrasound system 5 (on a monitor for example) in accordance with various aspects of the present invention. The various processors of the ultrasound machine 5 described above may be used to extract and display relevant summary information from various locations within the heart.

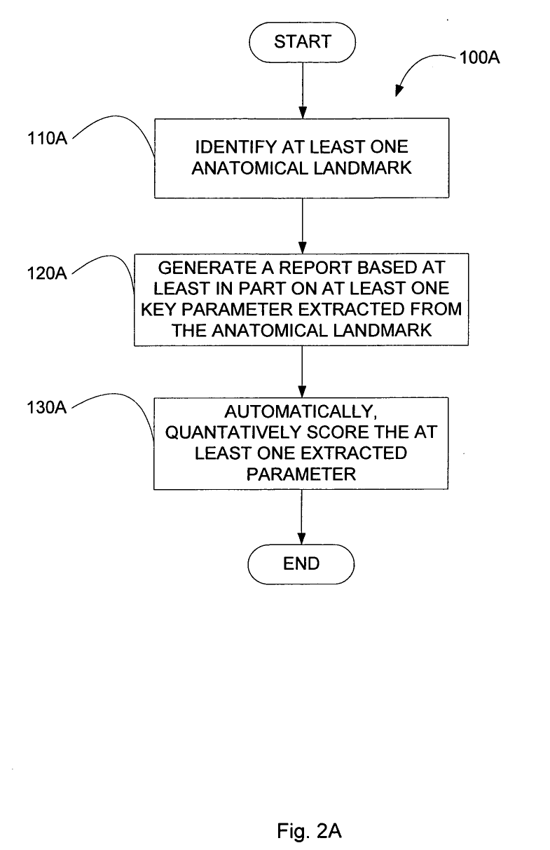

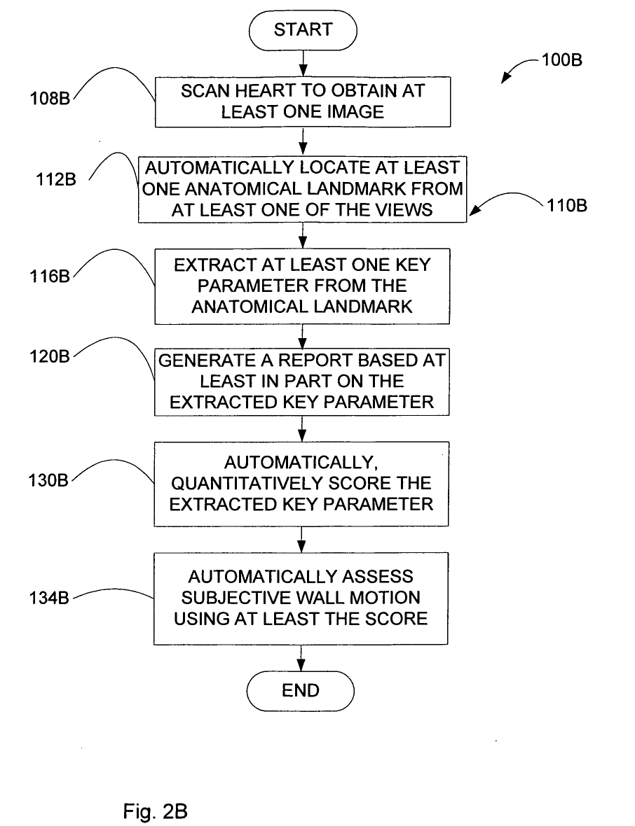

One embodiment of the present invention comprises a method for extracting ultrasound summary information useful for increasing efficiency of quantitation of stress echo examinations in accordance with various embodiments of the present invention. FIG. 2A depicts a high level flow chart illustrating a method 100A for extracting ultrasound summary information useful for increasing efficiency of quantitation of a stress echo examination. In the illustrated embodiment, the method 100A comprises Step 110A, identifying at least one anatomical landmark (e.g., the AV-plane and apex) within the heart while imaging the heart. Step 120A comprises generating a report based, at least in part, on the at least one key parameter extracted from the positions of the anatomical landmarks. Step 130A comprises quantitatively scoring (automatically in one embodiment) the at least one extracted parameter.

FIG. 2B depicts a flow chart illustrating an embodiment of a method 100B (similar to method 100A of in FIG. 2A) performed using a machine 5 illustrated in FIG. 1 for example in accordance with various aspects of the present invention. Again it is contemplated that the method 100B comprises identifying anatomical landmarks within the heart while imaging the heart, generally designated 110B. 110B may comprise Step 108B, scanning the heart to obtain one or more apical images. Step 112B comprises locating (automatically for example) at least one anatomical landmark from at least one of the views. In one embodiment, locating the anatomical landmark may further comprise automatically locating the AV-plane and apex, and automatically marking the AV-plane and apex with indicia and tracking, forming at least one anatomical landmark.

Method 100B may further comprise Step 116B, extracting at least one key parameter (also referred to as relevant summary information) from the anatomical landmark. Step 120B comprises generating a report based, at least in part, on the at least one key parameter (or relevant ultrasound summary information).

As defined herein, relevant ultrasound summary information comprises at least one of Doppler profile information (i.e., over time), velocity profile information, strain rate profile information, strain profile information, M-mode information, deformation information, displacement information, and B-mode information.

Method 100B further comprises Step 130B, scoring the at least one extracted key parameter. In one embodiment, the key parameter is automatically quantitatively scored using the ultrasound machine in accordance with one embodiment of the present invention. Method 100B further comprises Step 134B, assessing the wall motion using the score for example. In one embodiment, the wall motion is atomically assessed using the ultrasound machine in accordance with one embodiment of the present invention.

FIG. 3 depicts a diagram using methods 100A and 100B illustrated in FIGS. 2A and 2B respectively, to identify at least the lower parts of basal segments and mid segments within a heart in accordance with at least one embodiment of the present invention. Detected landmarks may be used to identify locations within the heart provided by relative positioning and local image characteristics. FIG. 3 illustrates two depictions of a heart 300. An image of the heart 300 with various markers overlaying certain anatomical locations is shown on the left of FIG. 3. A graphical illustration of the heart 300 with various markers overlaying certain anatomical locations is shown on the right of FIG. 3. FIG. 3 further provides an example in which the lower parts of myocardium in the basal segments 301 of the heart 300 and the lower part of the mid segments 302 of the heart 300 are identified relative to the detected landmarks (i.e.; apex 303 and AV-plane 304).

In accordance with at least one embodiment of the present invention, a region-of-interest (ROI) may be preset with respect to the anatomical landmarks. The extracted information may include one or more of Doppler information over time, velocity information over time, strain rate information over time, strain information over time, M-mode information, deformation information, displacement information, and B-mode information. Further, indicia may be overlaid onto the anatomical landmarks to clearly display the positions of the landmarks.

FIG. 4 depicts a diagram illustrating normal peak values at peak exercise in dobutamine stress in an apical 4-chamber view of a heart 400. The basal segments 401 and 402 are at about 13 cm/second, while the mid segments 403 and 404 are at about 10-11 cm/second. The associated percentage changes indicate the relative change in peak systolic velocity relative to corresponding rest values.

FIG. 5 depicts a diagram (similar to the diagram of FIG. 4) illustrating normal peak values at peak exercise in dobutamine stress in an apical 2-chamber view of the heart 500. The basal segments 501 and 502 are at about 9 cm/second, while and the mid segments 503 and 504 are at about 12-14 cm/second.

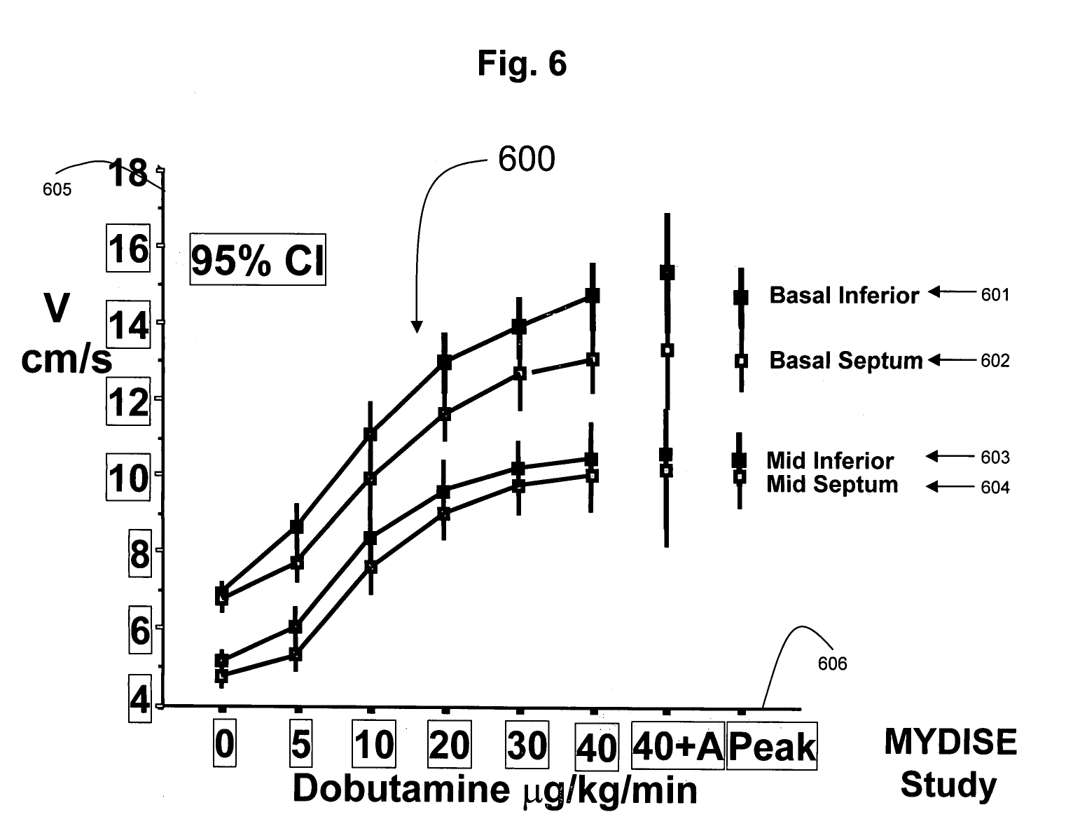

FIG. 6 depicts the response curves 600 for peak systolic velocities 605 as a function of dobutamin level 606 in a normal population. FIG. 6 depicts four locations that have been measured from apex. While four locations are depicted, more or different locations are contemplated. The four locations are basal inferior 601, basal septum 602, mid-inferior 603, and mid-septum 604. The depicted locations are able to approximately double the peak systolic velocity at peak exercise. Clinical results indicate that a reduction in peak systolic velocities at peak exercise is a good predictor of coronary artery diseases. The normal values illustrated in FIGS. 4-6 (or similar normal ranges) may be combined with other display techniques to indicate normal values both textually together with the measured parameters, or graphically as a normal scaling factor for the velocity or strain rate profiles.

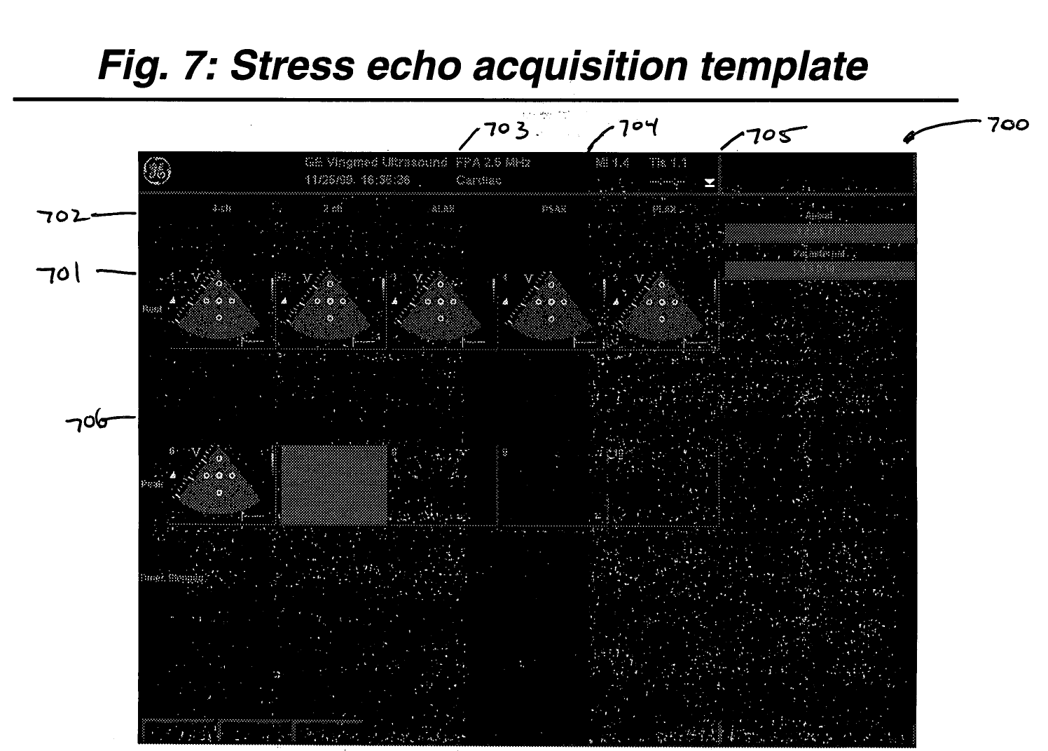

FIG. 7 depicts one method typically used to acquire and display stress echo, generally designated 700. A plurality of views (apical views designated 701, 702, 703, 704, 705, and 706) are collected at rest. It should be appreciated that while six views are illustrated, more or less views are contemplated. This process is repeated for a number of intermediate stress levels. Finally, a similar set of views is collected at peak exercise created due to stress. The stress may be caused by physical exercise or drugs using dobutamin.

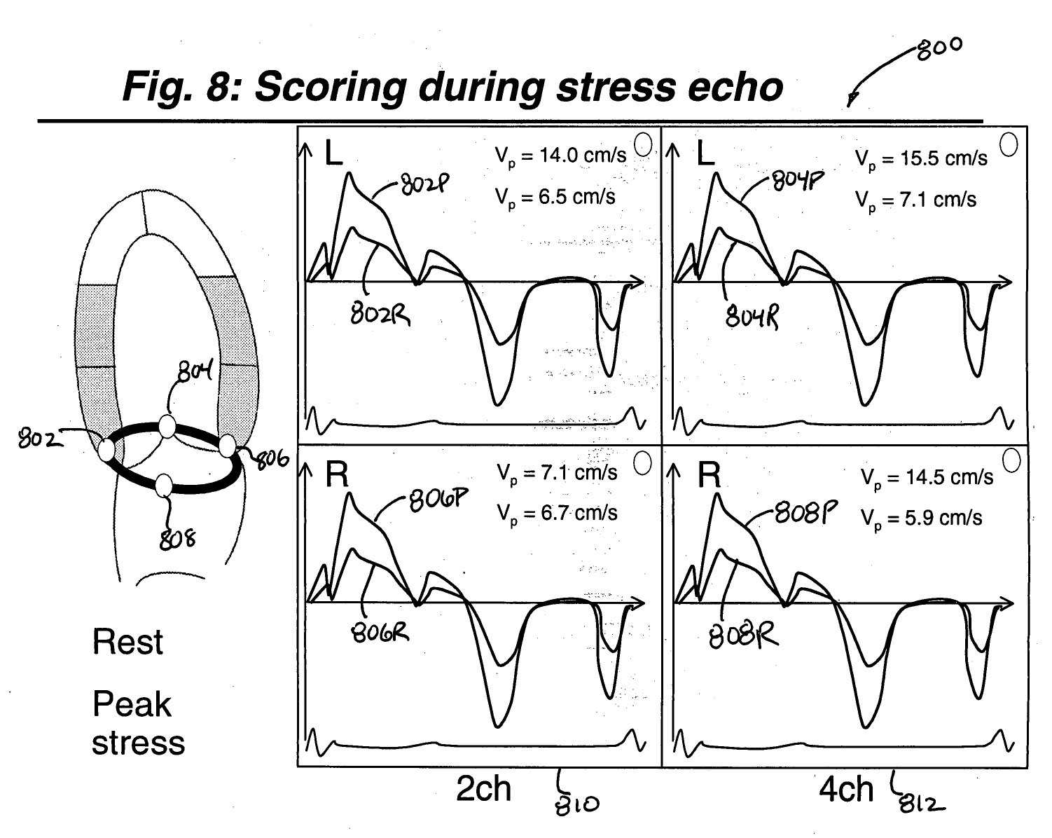

FIG. 8 illustrates detecting an AV-plane of the heart 800 (using an ultrasound device for example) that may be used to extract at least one key parameter (velocity profiles for example) and generate a basis for reporting, generally designated 800, in accordance with embodiments of the present invention. FIG. 8 depicts four AV-locations 802, 804, 806 and 808 extracted from apical 4-chamber and apical 2-chamber, designated 812 and 810 respectively. Each of these AV-locations are illustrated with the rest and peak velocity profiles and/or key extracted parameters. In one embodiment, the rest and peak velocity profiles are depicted graphically (802R, 804R, 806R, 808R, 802P, 804P, 806P and 808P) in addition to providing numeric values for each of the key parameters. It is contemplated that the extracted parameters may also be compared with normal ranges similar to those described previously in FIGS. 4-6. Similarly, 6 AV-plane locations may be quantified if an apical long axis view was used, in addition to the 4-chamber and 2-chamber views 802 and 804.

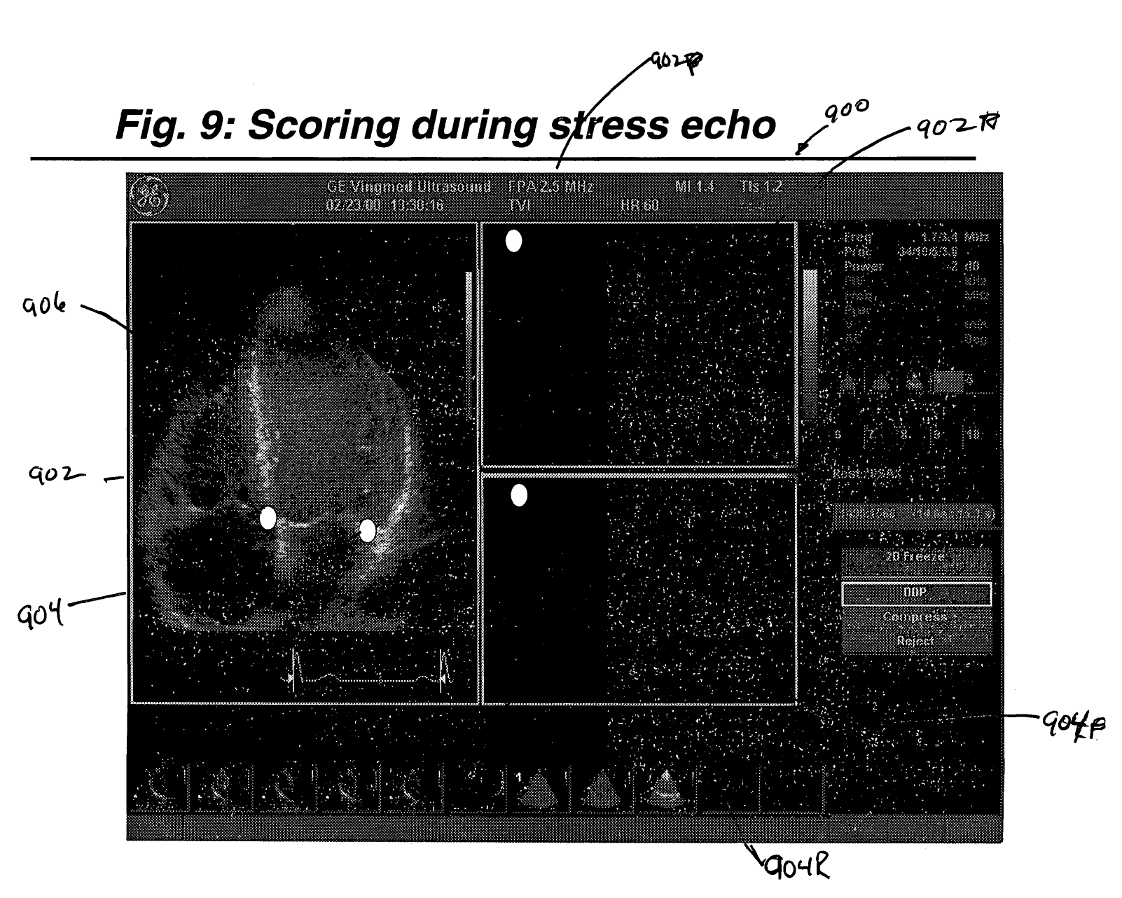

FIG. 9 depicts detecting the AV-plane used to extract at least one key parameter (velocity profiles for example) and generate a basis for reporting, generally designated 900, similar to that depicted in FIG. 8 in accordance with embodiments of the present invention. FIG. 9 depicts one method for displaying velocity information for 2 AV-plane locations, generally designated 902 and 904, including rest and peak velocity profiles and/or key extracted parameters. In one embodiment, the velocity information for the 2 AV-plane locations 902 and 904 are depicted graphically at rest and peak exercise (designated 902R, 904R, 902P and 904P) in addition to displaying numerical values, alone or in combination with a given 2D cineloop, generally designated 906. It is contemplated that either 2D cineloop for rest or peak activity may be depicted.

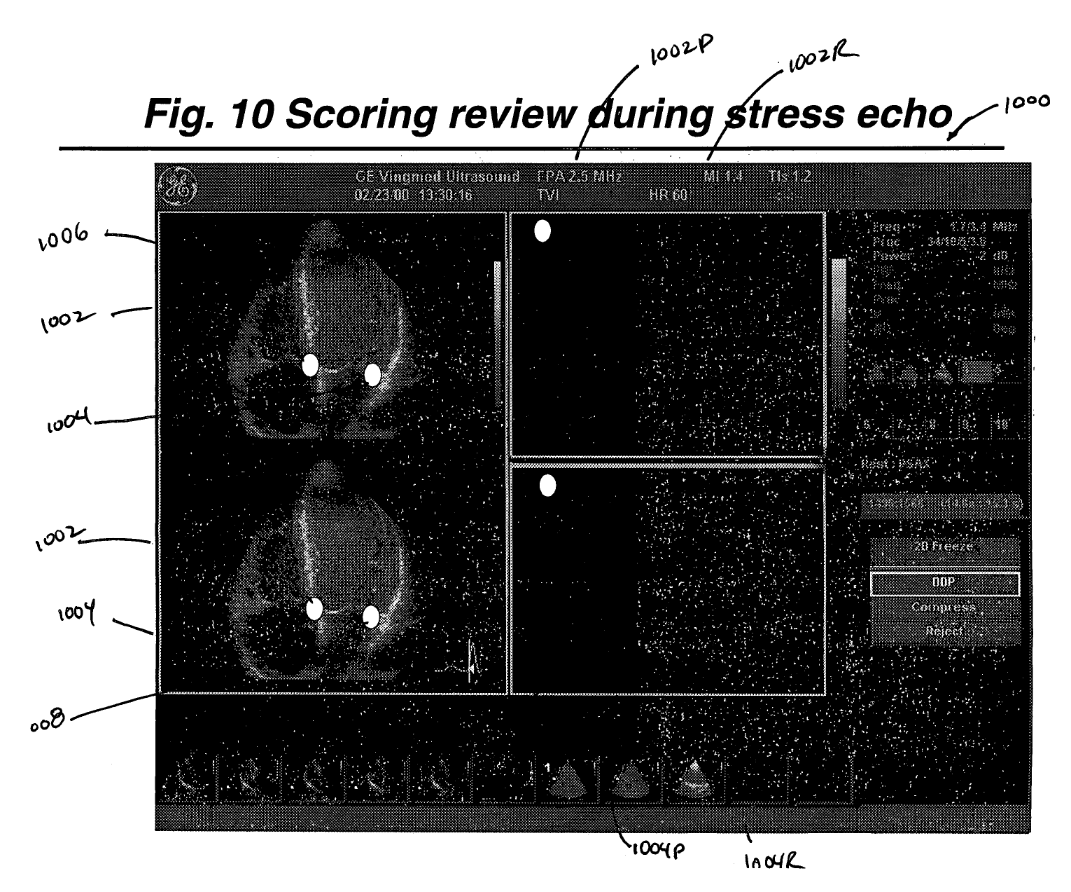

FIG. 10 depicts detecting the AV-plane used to extract at least one key parameter (velocity profiles for example) and generate a basis for reporting, generally designated 1000, similar to that depicted in FIG. 9 in accordance with embodiments of the present invention. FIG. 10 depicts one method for displaying velocity information for 2 AV-plane locations, generally designated 1002 and 1004, including rest and peak velocity profiles and/or key extracted parameters. In one embodiment, the velocity information for the 2 AV-plane locations 1002 and 1004 are depicted graphically at rest and peak exercise (designated 1002R, 1004R, 1002P and 1004P) in addition to numerical values, again alone or together with rest and peak exercise 2D cineloops 1006 and 1008 which, in this embodiment, are depicted simultaneously.

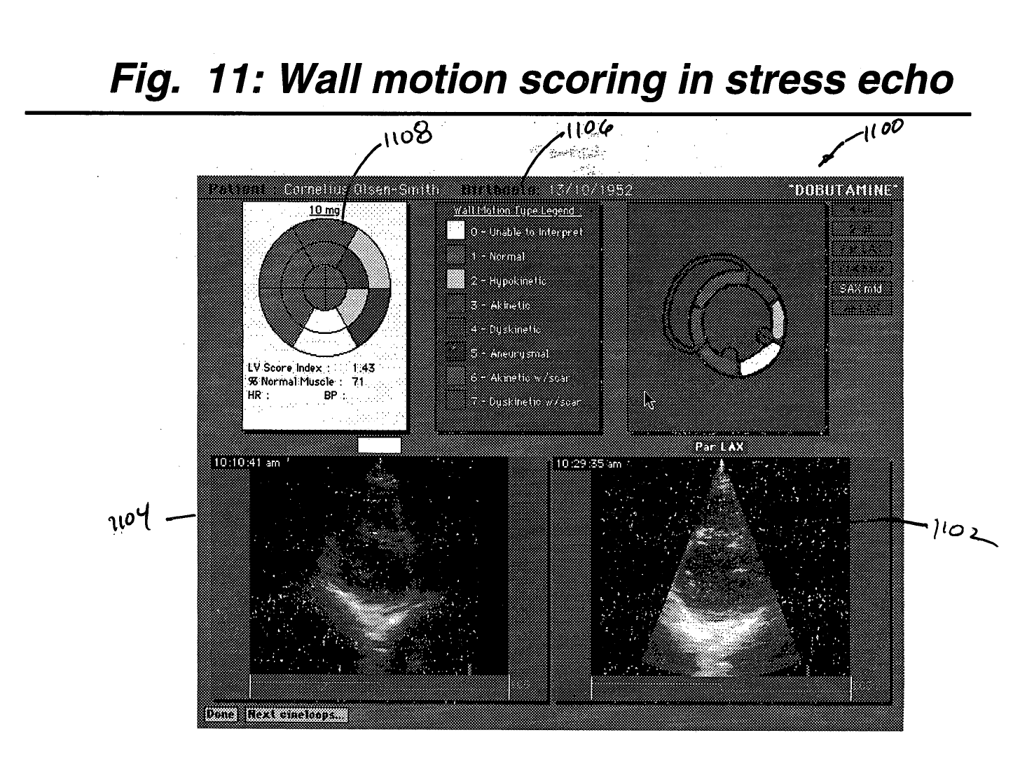

FIG. 11 depicts one known method for scoring subjective wall motion generally designated 1100. In such known method, two views 1102 and 1104 are compared visually. The user assigns his interpretation (i.e., scoring) to the associated wall segments using legend 1106, forming bulls-eye plot 1108.

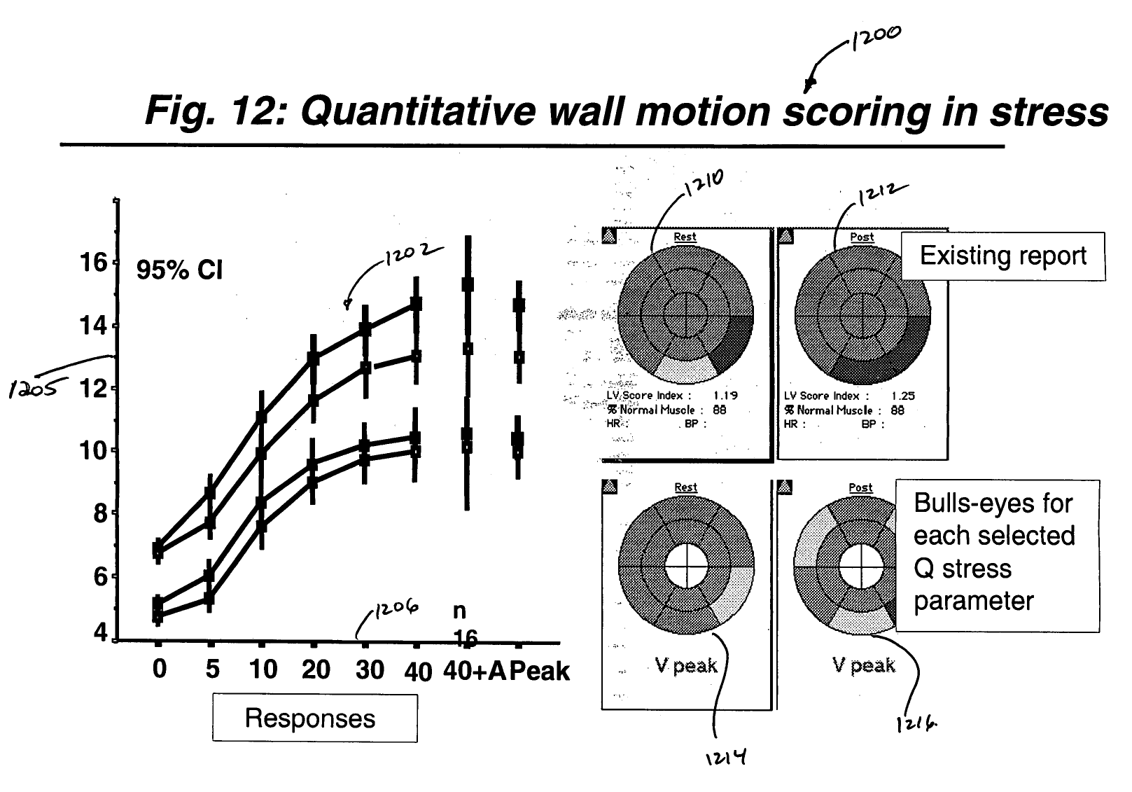

FIG. 12 depicts a method for augmenting subjective wall motion assessment using quantitative scoring, generally designated 1200, in accordance with embodiments of the present invention. In this embodiment, FIG. 12 depicts the response curves 1202 for peak systolic velocities 1205 as a function of dobutamin level 1206. FIG. 12 depicts four locations that have been measured from apex, similar to that depicted in FIG. 6. Again while four locations are depicted, more, less or different locations are contemplated.

FIG. 12 further depicts four bulls-eye plots 1210, 1212, 1214 and 1216. In this embodiment, bulls-eye plot 1212 depicts an existing report, while bulls-eye plot 1216 depicts scoring of a key parameter (peak systolic velocity for example) relative to predefined normal ranges. It should be appreciated that, while four bulls-eye plots (one plot for each extracted parameter) are depicted, more or less plots are contemplated.

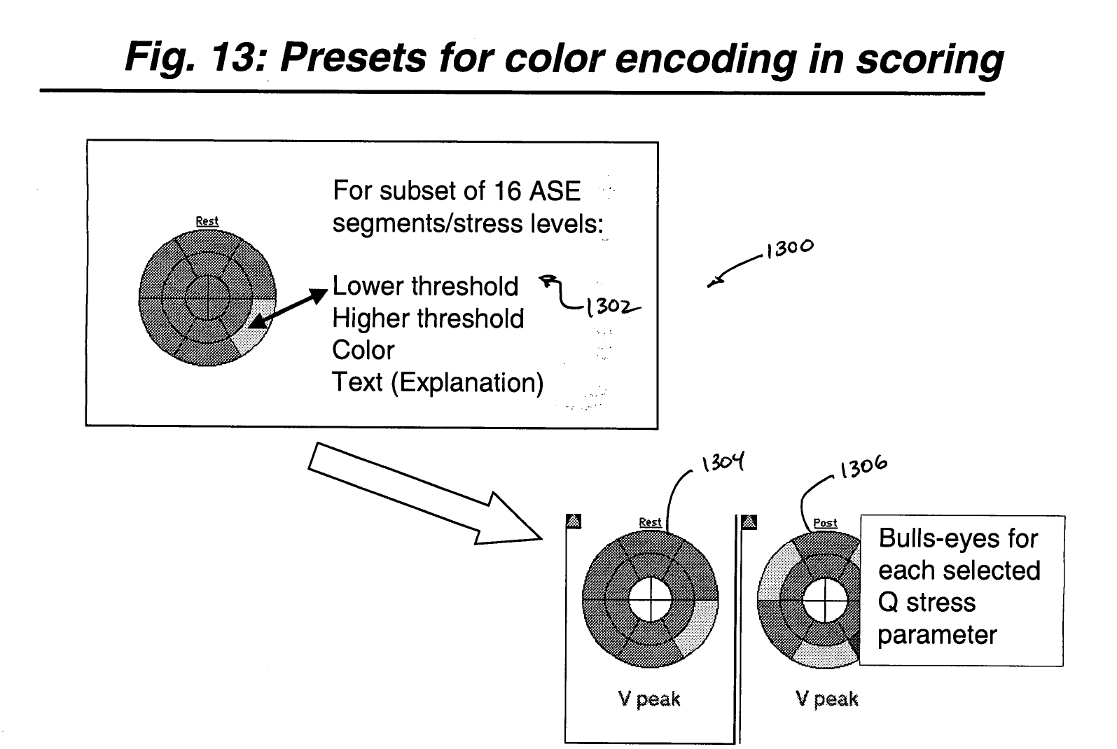

FIG. 13 depicts a method for displaying information about normal ranges of one or more key parameters, generally designated 1300, in accordance with embodiments of the invention. In this embodiment, normal ranges (lower threshold and upper threshold for a subset of 16 ASE segments/stress levels for example) and generally designated 1302 may each be assigned a predetermined color. A textual description may also be included. At least one bulls-eye plot may be generated for each selected key parameter. In this embodiment, two bulls-eye plots 1304 and 1306, are illustrated, one plot for the lower threshold and one for the higher threshold.

It should be appreciated that extracting one or more key parameters (velocity for example) at the AV-plane (or slightly above in the lower basal segment) provides a rough but sensitive assessment of cardiac function during stress. Such assessment corresponds to filling out the outer circle in the bulls-eye plots. In addition, this information may be supplemented with data from more locations collected either manually or using points related to extracted landmarks.

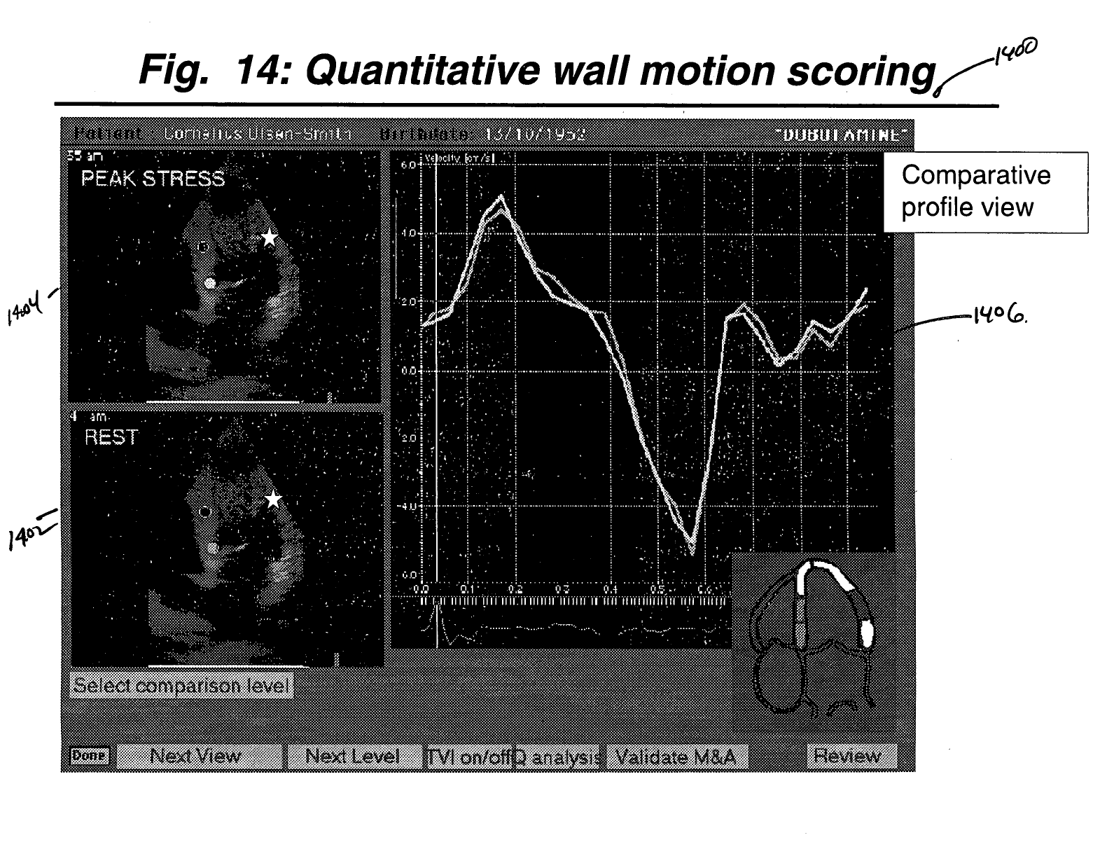

FIG. 14 depicts one method for performing quantitative wall motion scoring in accordance with one or more embodiments of the present invention. FIG. 14 illustrates one method, generally designated 1400, for extracting one or more key parameters (in this embodiment velocity profiles) from corresponding locations in rest and exercise cineloops (two locations 1402 and 1404 are illustrated in the loops) The information is provided in graph 1406.

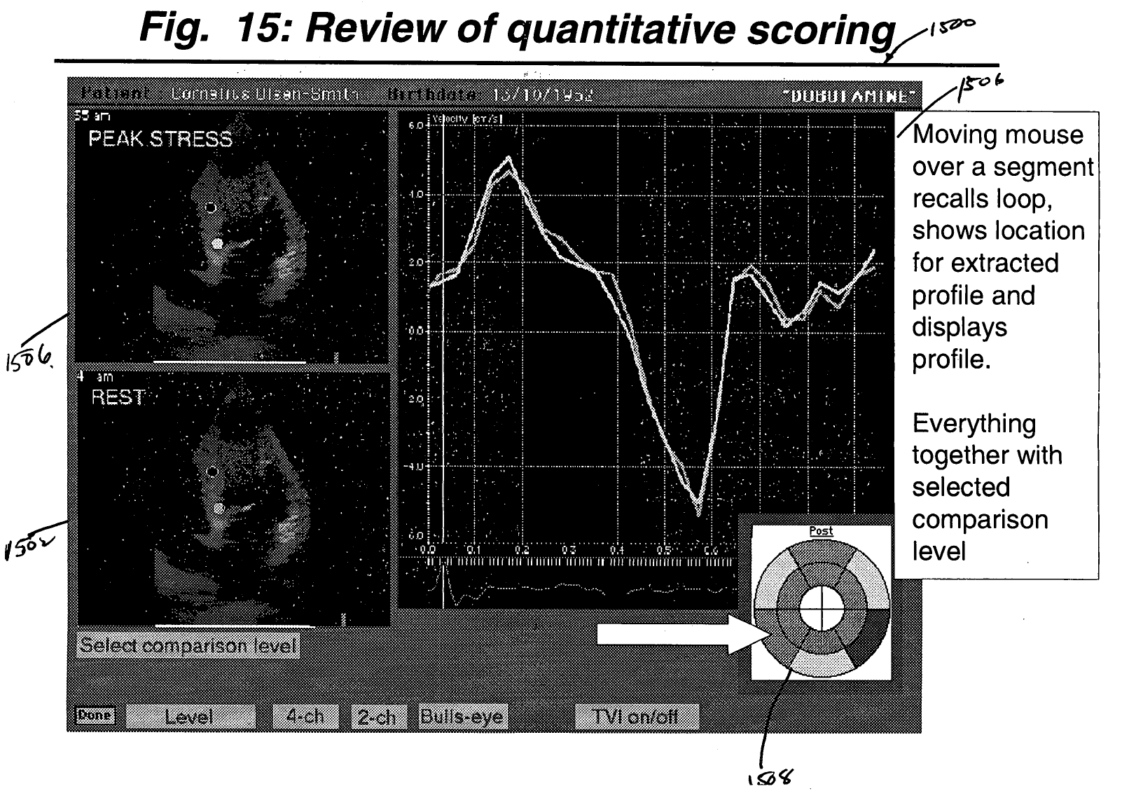

FIG. 15 depicts one method for using a scoring screen to review quantitative scoring, generally designated 1500, acquired using a method similar to that illustrated in FIG. 14. In this embodiment, a wall-segment 1506 in bulls-eye plot 1508 may be selected using a mouse, touchscreen or other similar device. Selecting the wall-segment, recalls the associated cineloops 1502 and 1504 (displaying the location for the extracted key profile) and displays the associated velocity profile 1506.

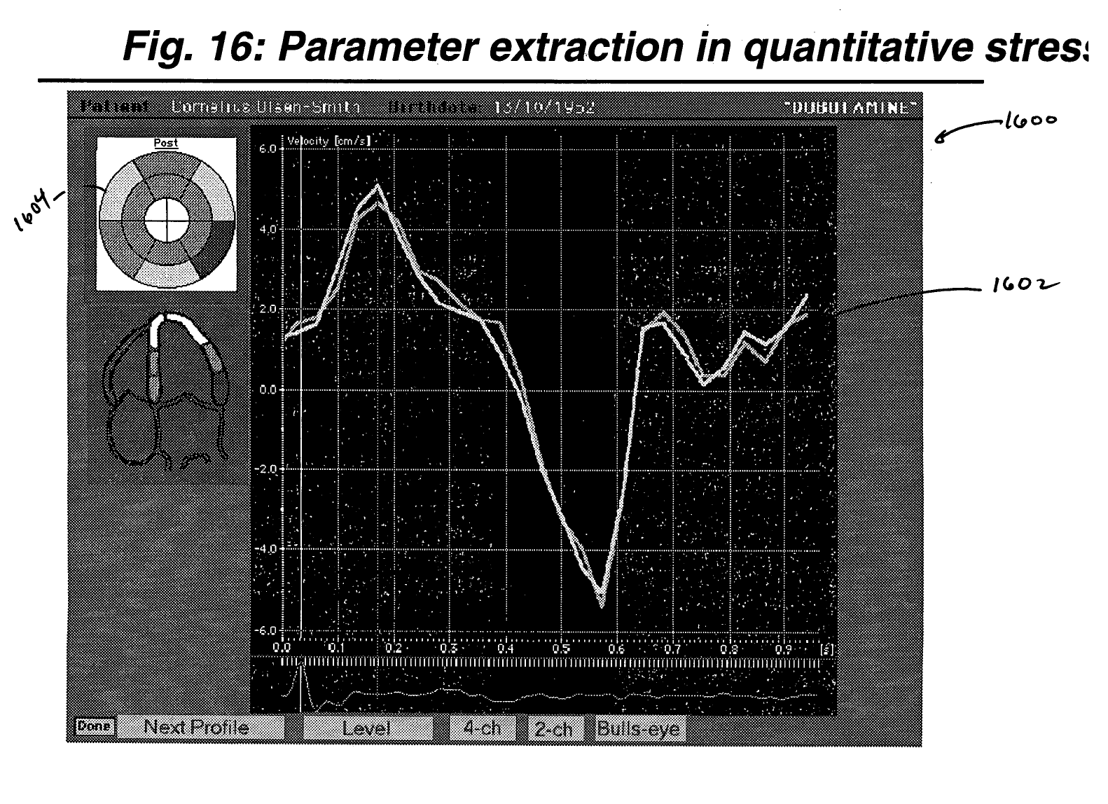

FIG. 16 depicts one method for automatically performing wall motion scoring, generally designated 1600, using an ultrasound machine similar to that provided previously. In at least one embodiment, one or more key parameters (peak systolic velocity for example) may be algorithmically extracted from at least one velocity profile 1602. The algorithmically extracted key parameter may be scored and plotted, generally designated 1604. In at least one embodiment, the system and method efficiently enables a user to review how the extracted parameters have been computed. In this embodiment, the user may override mistakes made by the automatic algorithms.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for assessing an image responsive to moving cardiac structure and blood within a heart of a subject, the method comprising:
identifying at least one anatomical landmark within the heart;
generating a report based at least in part on one key parameter extracted from said anatomical landmark; and
scoring said at least one extracted parameter.

2. The method of claim 1 comprising using an ultrasound machine to identify said anatomical landmark, generate said report and score said at least one parameter.

3. The method of claim 1 comprising displaying at least said scoring.

4. The method of claim 1 wherein identifying said at least one anatomical landmark comprises acquiring at least one apical view of the heart.

5. The method of claim 1 wherein identifying said at least one anatomical landmark comprises automatically identifying an AV-plane of the heart.

6. The method of claim 1 comprising automatically quantitatively scoring said at least one extracted parameter.

7. The method of claim 6 comprising algorithmically extracting said at least one key parameter.

8. The method of claim 6 comprising selecting at least a portion of said quantitatively scored extracted parameter and displaying at least an associated velocity profile of said extracted parameter.

9. In an ultrasound machine for generating an image responsive to moving cardiac structure and blood within a heart of a subject, a method comprising:
acquiring at least one apical view of the heart using the ultrasound machine;
generating at least one image of said apical view on a display of the ultrasound machine;
automatically identifying at least one anatomical landmark from at least one of said views using the ultrasound machine;
generating a report based at least in part on one key parameter extracted from said anatomical landmark; and
scoring said at least one extracted parameter.

10. The method of claim 9 wherein identifying said at least one anatomical landmark comprises automatically identifying an AV-plane of the heart.

11. The method of claim 9 comprising quantitatively scoring said at least one extracted parameter.

12. The method of claim 11 comprising selecting at least a portion of said quantitatively scored extracted parameter and displaying at least an associated velocity profile of said extracted parameter.

13. The method of claim 9 comprising automatically quantitatively scoring said at least one extracted parameter.

14. The method of claim 13 comprising algorithmically extracting said at least one key parameter.

15. The method of claim 9 comprising displaying said score on said display of said ultrasound machine.

16. The method of claim 9 wherein said at least one anatomical landmark comprises at least one of an apex of said heart and an AV-plane of said heart.

17. In an ultrasound machine for generating an image responsive to moving cardiac structure and blood within a heart of a subject, an apparatus comprising:
a front-end arranged to transmit ultrasound waves into said moving cardiac structure and blood, generating received signals in response to the ultrasound waves backscattered from said moving cardiac structure and blood;
at least one processor responsive to the received signals, identifying at least one anatomical landmark within the heart, generating a report based at least in part on one key parameter extracted from said anatomical landmark, and scoring said at least one extracted parameter.

18. The apparatus of claim 17 further comprising a display processor and monitor adapted to display at least said scoring of said extracted parameter.

19. The apparatus of claim 17 wherein the at least one processor comprises at least one of a Doppler processor, a non-Doppler processor, a control processor, and a PC back-end.

20. The apparatus of claim 17 further comprising at least one user interface connecting to said at least one processor to control operation of said ultrasound machine.
